(19) United States
(12) Patent Application Publication
(10) Pub. No.: US 2021/0161506 A1
(43) Pub. Date: Jun. 3, 2021

(54) ULTRASOUND DIAGNOSTIC APPARATUS AND DISPLAY METHOD

(71) Applicant: Hitachi, Ltd., Tokyo (JP)

(72) Inventor: Takumi Ito, Tokyo (JP)

(73) Assignee: Hitachi, Ltd., Tokyo (JP)

(21) Appl. No.: 16/895,458

(22) Filed: Jun. 8, 2020

(30) Foreign Application Priority Data
Nov. 28, 2019 (JP) 2019-214813

(51) Int. Cl. A61B 8/08; G01N 29/32; G01N 29/24; A61B 8/00; G06T 7/00

(52) U.S. Cl. CPC A61B 8/0825; G01N 29/32; G01N 29/24; G06T 2207/30068; A61B 8/5215; A61B 8/461; G06T 7/0012; A61B 8/5207

(57) ABSTRACT

A probe operation is supported in ultrasound diagnosis of a breast. A tomographic image includes a mammary gland image, a pectoralis major image, and a boundary image between the mammary gland image and the pectoralis major image. By detection of the boundary image, an approximate straight line approximating the boundary image is calculated. An inclination angle of the approximate straight line is calculated. When the inclination angle exceeds a threshold, a support image showing that a probe contact posture is inappropriate is displayed. When the inclination angle is less than or equal to the threshold, a support image showing that the probe contact posture is appropriate is displayed. When the inclination angle exceeds the threshold, a CAD function may be stopped.

### CROSS REFERENCE TO RELATED APPLICATION

This application claims priority to Japanese Patent Application No. 2019-214813 filed on Nov. 28, 2019, which is incorporated herein by reference in its entirety including the specification, claims, drawings, and abstract.

### TECHNICAL FIELD

The present invention generally relates to an ultrasound diagnostic apparatus, and more particularly to a technique for supporting a probe operation.

### BACKGROUND

An ultrasound diagnostic apparatus is an apparatus that forms an ultrasound image based on a reception signal obtained by transmitting and receiving ultrasonic waves to and from a living body. The ultrasound image is, for example, a tomographic image, which is an image that shows a section of a tissue. For example, in the examination of a breast, an ultrasound probe is brought into contact with the surface of the breast, a tomographic image displayed thereby is observed, and through observation of the tomographic image, the presence or absence of a tumor in the mammary gland, the mode of the tumor, and the like are diagnosed.

In recent years, an ultrasound diagnostic apparatus, and an ultrasound image processing apparatus equipped with computer-aided diagnosis (Computer-Aided Diagnosis: CAD) functions have been widely used. In these apparatuses, CAD functions are used in estimation or diagnosis of ultrasound images. For example, in a mammary gland diagnosis, a tomographic image is analyzed in real time by means of a CAD function. More specifically, a low-luminance tumor image (or a low-luminance non-tumor) included in the tomographic image is automatically recognized, and is marked. Some CAD functions automatically determine the degree of malignancy for each tumor image. JP 2015-54007 A discloses an ultrasound diagnostic apparatus that detects probe posture deviation. Special circumstances in ultrasound diagnosis of breasts are not considered in the ultrasound diagnostic apparatus.

### SUMMARY

### Technical Problem

In ultrasound diagnosis of a breast, it is necessary to correctly apply a probe to each diagnostic position on the breast. When the probe is not correctly applied, many unclear portions occur in the ultrasound image, and shadows occur in the end portions of the ultrasound image. The breast is a soft body with a bulge. The shapes and sizes of breasts vary considerably from subject to subject, and the shape of the breast changes greatly depending on the posture of the subject. Unlike the case where the probe is brought into contact with a flat body surface, special attention is required when the probe is brought into contact with the breast. For example, a special probe operation may be required in which a mammary gland is sandwiched between the probe and the pectoralis major and the mammary gland is spread horizontally along the pectoralis major. It is not easy for an inexperienced operator to always make appropriate probe contact posture to the breast.

An advantage of the present disclosure is to support ultrasound diagnosis of breasts. Alternatively, it is an advantage of the present disclosure to provide a user with information indicating whether or not a probe contact posture to a breast is appropriate.

### Solution to Problem

An ultrasound diagnostic apparatus according to the present disclosure includes a probe that is brought into contact with a breast, and outputs a reception signal by transmission and reception of ultrasonic waves to and from the breast, an image generating unit that, based on the reception signal, generates an ultrasound image including a mammary gland image, a pectoralis major image, and a boundary image between the mammary gland image and the pectoralis major image, an inclination angle calculating unit that, based on the ultrasound image, calculates an inclination angle of the boundary image, and support image generating unit that generates a support image that supports an operation of the probe, based on the inclination angle of the boundary image.

A display method according to the present disclosure includes the steps of calculating an inclination angle of a boundary image, based on an ultrasound image including a mammary gland image, a pectoralis major image, and the boundary image between the mammary gland image and the pectoralis major image, generating a support image that supports an operation of a probe contacting a breast, based on the inclination angle of the boundary image, and displaying the support image.

### BRIEF DESCRIPTION OF DRAWINGS

Embodiment of the present disclosure will be described based on the following figures, wherein:

FIG. 1 is a block diagram showing an example configuration of an ultrasound diagnostic apparatus according to an embodiment;

FIG. 2 is a block diagram showing example configurations of an inclination angle calculating unit and an operation support image generating unit;

FIG. 3 is a diagram showing an example of a tomographic image;

FIG. 4 is a diagram showing a generation method of an approximate straight line;

FIG. 5 is diagram for explaining exclusion processing;

FIG. 6 is a diagram showing a boundary point row before and after smoothing;

FIG. 7 is a diagram for explaining a smoothing method;

FIG. 8 is a diagram showing a first example of a support image;

FIG. 9 is a diagram showing a second example of the support image:

FIG. 10 is a diagram showing a third example of the support image;

FIG. 11 is a diagram showing a fourth example of the support image;

FIG. 12 is a flowchart showing an operation example; and

FIG. 13 is a flowchart showing another operation example.

### DESCRIPTION OF EMBODIMENT

Hereinafter, an embodiment will be described based on the figures.

### (1) Outline of Embodiment

An ultrasound diagnostic apparatus according to the embodiment has a probe, an image generating unit, an inclination angle calculating unit, and a support image generating unit. The probe is brought into contact with a breast, and outputs a reception signal by transmission and reception of ultrasonic waves to and from the breast. Based on the reception signal, the image generating unit generates an ultrasound image including a mammary gland image, a pectoralis major image, and a boundary image between the mammary gland image and the pectoralis major image. The support image generating unit generates a support image that supports an operation of the probe based on an inclination angle of the boundary image.

According to the above described configuration, in ultrasound diagnosis of a breast, in particular, a mammary gland, a support image can be provided to an operator (user) operating the probe. Through observation of the support image, the user can easily judge whether or not the probe operation; in particular, a probe contact posture to the breast, is appropriate.

As described above, when the probe is brought into contact with the breast correctly in the ultrasound diagnosis of the breast, a boundary image becomes horizontal or nearly horizontal in a tomographic image. More specifically, in a state where a relatively soft mammary gland is sandwiched between the wave transmission and reception surface of the probe and a surface of the pectoralis major, and at the same time, a substantially uniform pressing force is applied to the entire mammary gland, the wave transmission and reception surface and the surface of the pectoralis major become parallel or nearly parallel. Through provision of the support image, the above-described configuration informs the user whether or not the boundary image is nearly horizontal; that is, whether or not a probe contact posture is appropriate.

In the embodiment, the support image is displayed with an ultrasound image. The support image may be displayed by being superimposed on the ultrasound image, or the support image may be displayed around the ultrasound image. The ultrasound image and the support image may be separately displayed on two displays. Another kind of information (for example, sound information) indicating that the probe contact posture is appropriate or inappropriate may be provided to the user with the display information (or instead of the display information).

In the embodiment, the support image is displayed in real time. In other words, the support image is displayed as a moving image in a process of displaying an ultrasound image as a moving image. Thereby, a probe operation is supported in real time. As a matter of course, the support image may be displayed as reference information in the process of reproducing an ultrasound image after freezing. A concept of the probe can encompass a general-purpose probe, a breast examination probe, and the like.

The ultrasound diagnosis apparatus according to the embodiment further includes a determining unit. The determining unit determines inappropriateness concerning a probe contact posture, based on the inclination angle of the boundary image. The user is informed of inappropriateness through the support image. According to the configuration, the user can clearly recognize that the probe contact posture becomes inappropriate. In the case where the probe contact posture is appropriate and in the case where the probe contact posture is inappropriate, it may be arranged such that the support image is displayed only in the latter case, or such that the support image is displayed in both cases. When the support image is always displayed, a display mode of the support image is changed according to appropriateness and inappropriateness of the probe contact posture. A degree of inappropriateness may be determined stepwise or continuously.

In the embodiment, the determination unit determines inappropriateness when the inclination angle exceeds a threshold. The ultrasound diagnostic apparatus according to the embodiment further includes a threshold setting unit that changes a threshold according to a depth of the boundary image. The configuration changes an allowable range of the inclination angle of the boundary image according to a probe contact position on a breast, a size of the breast, a shape of the breast, and the like. According to the configuration, excessively strict determination can be avoided, for example.

In the embodiment, the inclination angle calculating unit has a generator and a calculator. The generator generates an approximate straight line based on the boundary image. The calculator calculates an intersection angle of the approximate straight line to a horizontal direction, as an inclination angle. The inclination angle may be directly calculated from the boundary image without obtaining the approximate straight line. When the approximate straight line is generated in a form of a function, a part that derives the function corresponds to both the generator and the calculator.

In the embodiment, the generator sets a plurality of search routes to cross the boundary image, performs boundary search from a deep side to a shallow side on each of the search routes to specify a boundary point, and generates the approximate straight line based on a plurality of boundary points specified on the plurality of search routes. Generally, luminance is uniformly low in an inside of a pectoralis major image on an ultrasound image. Therefore, it becomes possible to determine boundary points correctly when the boundary search is performed from a deeper side of the boundary image to a shallow side.

In the embodiment, the generator specifies, among a plurality of boundary points, a plurality of effective boundary points by excluding invalid boundary points satisfying an exclusion condition, and calculates an approximate straight line based on the plurality of effective boundary points. A lesion site such as a tumor may occur on the boundary image, or some of the boundary points may be specified at inappropriate positions due to an effect of artifacts and the like. It becomes possible to obtain an approximate straight line more accurately by excluding an invalid boundary point satisfying the exclusion condition, and calculating the approximate straight line based on the plurality of effective boundary points. A lower side of a region of interest (ROI) to be a target of image analysis may be defined by the approximate straight line or a boundary image trace line.

The ultrasound diagnosis apparatus according to the embodiment includes an analyzing unit and a control unit. The analyzing unit searches for an abnormal site in an ultrasound image. The control unit limits an operation of the analyzing unit based on the inclination angle of the boundary image. When the inclination angle of the boundary image is large, relatively many artifacts occur in the ultrasound image, and there is a high possibility that the artifacts are erroneously recognized as a lesion site. For example, when there exists a part where a degree of close contact of the wave reception surface and the breast surface is low, a part of the ultrasound image is missing; that is, a shadow occurs. There is a high possibility that the shadow like this is erroneously recognized as a lesion site. The above described configuration limits the operation of the analyzing unit and prevents provision of erroneous information to the user, when reduction in quality of the ultrasound image is predicted. A threshold for determining that the probe contact posture is inappropriate and a threshold for limiting the operation of the analyzing unit may be provided separately.

The display method according to the embodiment has an inclination angle calculating step, a support image generating step, and a display step. In the inclination angle calculating step, the inclination angle of the boundary image is calculated based on an ultrasound image including a mammary gland image, a pectoralis major image, and a boundary image between the mammary gland image and the pectoralis major image. In the support image generating step, a support image that supports an operation of the probe contacting the breast is generated based on the inclination angle of the boundary image. In the display step, the support image is displayed. According to the configuration, it is possible to confirm correctness of the probe contact posture, or it is possible to recognize that the probe contact posture is incorrect through observation of the support image.

The above-described method can be realized as a function of hardware or as a function of software. In the case of the latter, a program for executing the above-described method is installed to an information processing apparatus via a network or a portable storage medium. A concept of the information processing apparatus encompasses an ultrasound diagnostic apparatus, an ultrasound diagnostic system, and the like. The information processing apparatus includes a processor such as a CPU, and the processor exhibits the respective functions described above.

### (2) Details of Embodiment

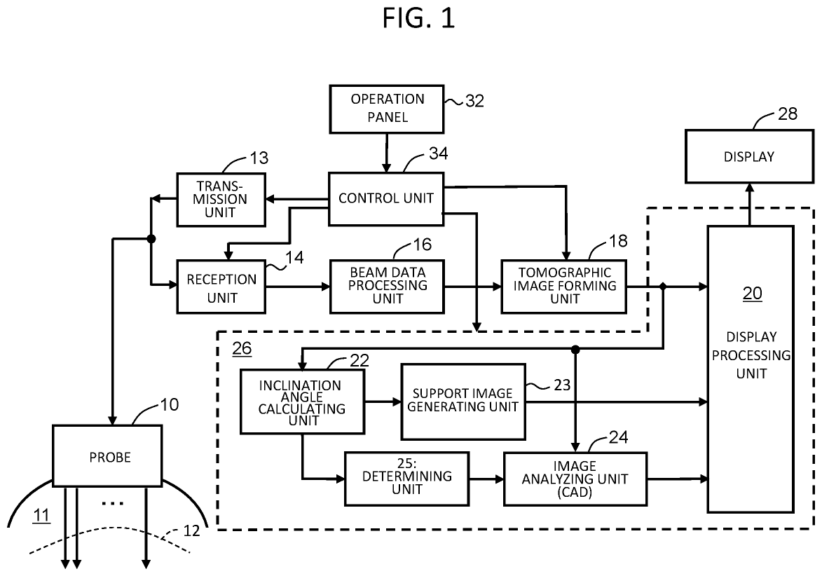

FIG. 1 shows a configuration of the ultrasound diagnostic apparatus according to the embodiment as a block diagram. The ultrasound diagnostic apparatus is a medical apparatus installed in a medical institution such as a hospital, and forms an ultrasound image based on a reception signal obtained by transmission and reception of ultrasonic waves to and from a living body (subject). The ultrasound diagnostic apparatus according to the embodiment includes a function of automatically analyzing an ultrasound image (CAD function), and a function of displaying information supporting the probe operation as described in detail later. In the embodiment, a tissue to be a target of ultrasound diagnosis is a breast; more specifically, a mammary gland.

A probe 10 functions as means that transmits and receives ultrasonic waves. The probe 10 is a portable transducer, and is held and operated by a user (a doctor, a laboratory technician, etc.). During ultrasound diagnosis of a mammary gland, a wave transmission and reception surface (acoustic lens surface) of the probe 10 is brought into contact with a surface of a breast 11 of a subject, and ultrasonic waves are transmitted and received in that state. In the breast 11, a mammary gland, a pectoralis major, and a boundary 12 between the mammary gland and the pectoralis major are present.

The ultrasound probe 10 includes a transducer element array formed of a plurality of transducer elements arranged one-dimensionally. An ultrasonic beam is formed by the transducer element array, and a scanning surface is formed by electronic scanning of the ultrasonic beam. The scanning surface is an observation surface; that is, a two-dimensional data acquisition area. As an electronic scanning method of ultrasonic beams, an electronic sector scanning method, an electronic linear scanning method, and the like are known. Convex scanning of ultrasonic beams may be performed. A two-dimensional transducer element array may be provided in the ultrasound probe, and volume data may be acquired from an inside of a living body.

A transmission unit 13 is a transmission beam former that supplies a plurality of transmission signals to a plurality of transducer elements in parallel during a transmission time, and is configured as an electronic circuit. A reception unit 14 is a reception beam former that phases and adds (delays and sum) a plurality of reception signals outputted from the plurality of transducer elements in parallel during a reception time, and is configured as an electronic circuit. The reception unit 14 includes a plurality of A/D converters, a wave detection circuit, and the like. Beam data are generated by phasing and addition of the plurality of reception signals in the reception unit 14. Incidentally, a plurality of sets of beam data arranged in an electron scanning direction are generated per electronic scanning of one time, and these sets of beam data configure reception frame data. Individual beam data are configured by a plurality of sets of echo data arranged in a depth direction.

A beam data processing unit 16 is an electronic circuit that processes respective beam data outputted from the reception unit 14. The processing includes logarithmic transformation, correlation processing, and the like. The respective beam data after processing are sent to a tomographic image forming unit 18.

The tomographic image forming unit 18 is an electronic circuit forming a tomographic image (B-mode tomographic image) based on the reception frame data. The tomographic image forming unit 18 has a DSC (Digital Scan Converter). The DSC has a coordinate conversion function, an interpolation function, a frame rate conversion function, and the like, and forms a tomographic image based on the reception frame data formed of a plurality of sets of beam data arranged in a beam scanning direction. Data of a tomographic image are sent to a display processing unit 20 and an inclination angle calculating unit 22.

Note that in the embodiment, the display processing unit 20, the inclination angle calculating unit 22, a support image generating unit 23, a determining unit 25, and an image analyzing unit 24 that are described below configure an image processing module 26. The image processing module 26 can be configured by one or a plurality of processors operating in accordance with a program. A CPU configuring a control unit 34 may function as the image processing module 26.

The inclination angle calculating unit 22 calculates an inclination angle of a boundary image included in a tomographic image. As described later, the tomographic image includes a mammary gland image and a pectoralis major image. The boundary image is present between the mammary gland image and the pectoralis major image, and is a linear image running in a substantially lateral direction. The inclination angle is an angle to the horizontal direction, and in the embodiment is an absolute angle having no sign.

The support image generating unit 23 generates a support image (probe operation support image) for supporting a probe operation by a user, based on the inclination angle. A display mode of the support image is an alert mode when the inclination angle exceeds a threshold, and is a non-alert mode when the inclination angle is less than or equal to the threshold. It may be the case that the support image is displayed only when the inclination angle exceeds the threshold. Similarly to the tomographic images, the support image is a moving image, and is displayed in real time. Data of the generated support image are sent to the display processing unit 20.

The image analyzing unit 24 functions as image analyzing means, and executes image analysis to an image part included in the region of interest in the tomographic image. In other words, the image analyzing unit 24 exhibits a CAD function. The image analyzing unit 24 performs image analysis in frame units. As a matter of course, image analysis may be executed with a predetermined number of frames as a unit. The image analyzing unit 24 can be configured by a machine learning type analyzer such as a CNN (Convolutional Neural Network). The image analyzing unit 24 has a function of recognizing, extracting, or discriminating a low-luminance tumor, a low-luminance non-tumor, and the like. The image analyzing unit 24 may include a function of evaluating a degree of malignancy of a tumor. In the embodiment, the image analyzing unit 24 analyzes a tomographic image and specifies a tumor or the like, and generates a marker that indicates the tumor or the like. An image analysis result including the marker is sent to the display processing unit 20. The image analyzing unit 24 basically operates in real time. As a matter of course, analysis may be performed on a reproduced tomographic image. In the image analyzing unit 24, processing may be performed parallel to a direction orthogonal to the approximate straight line, based on the calculated inclination angle.

The determining unit 25 controls the CAD function to turn on/off, based on the inclination angle. More specifically, the CAD function is brought into an on state when the inclination angle is within a threshold, and the CAD function is turned off when the inclination angle exceeds the threshold. Instead of the CAD function itself being turned on/off, display of a CAD result may be turned on/off. When the inclination angle is large, deterioration of tomographic image quality is predicted; in other words, there is a high possibility that relatively many artifacts appear in the tomographic image, or there is a high possibility that a degree of close contact of an end portion of the wave transmission and reception surface reduces and a shadow is generated; consequently, the CAD function is turned off from a viewpoint of prevention of error detection.

The display processing unit 20 has a graphic image generating function, a color calculating function, an image synthesizing function, and the like. More specifically, the display processing unit 20 generates a display image including a tomographic image, a support image, an image analysis result, and the like, and sends data of the display image to a display 28. The display 28 is configured by an LCD, an organic EL display device, or the like.

The control unit 34 controls operations of the respective components shown in FIG. 1. In the embodiment, the control unit 34 is configured by a CPU and a program. The control unit 34 may function as the above-described image processing module 26. An operation panel 32 is an input device, and has a plurality of switches, a plurality of buttons, a track ball, a keyboard, or the like. In FIG. 1, ultrasound image forming units other than the tomographic image forming unit 18 are not illustrated. For example, there may be provided an elasticity information (elastography) image forming unit, a blood flow image forming unit, and other units.

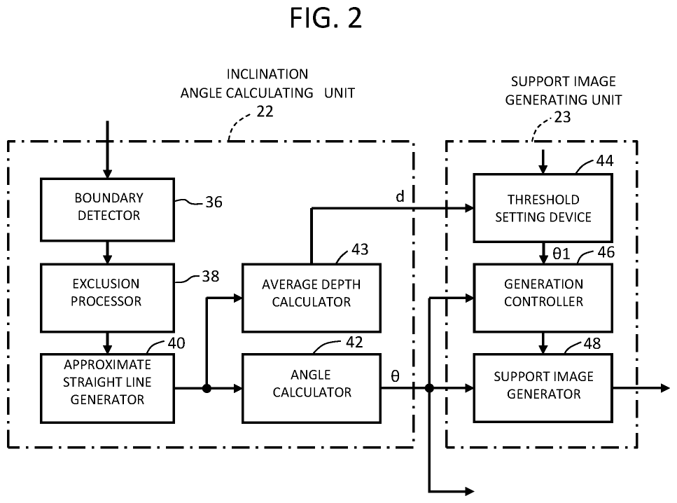

FIG. 2 shows example configurations of the inclination angle calculating unit 22 and the support image generating unit 23. The inclination angle calculating unit 22 has a boundary detector 36, an exclusion processor 38, an approximate straight line generator 40, an angle calculator 42, and an average depth calculator 43. The support image generating unit 23 has a threshold setting device 44, a generation controller 46, and a support image generator 48. The average depth calculator 43 is provided as necessary.

The boundary detector 36 sets a plurality of search routes on a tomographic image so as to cross the boundary image, and performs edge detection on each of the search routes. Thereby, a detection point line formed of a plurality of detection points that specify the boundary image is configured. The number of search routes to be set may be variably set by the user.

Prior to boundary detection, preprocessing is applied to the tomographic image. As the preprocessing, there are cited smoothing processing, minimum value extraction processing, maximum value extraction processing, median (median value extraction) processing, edge enhancement processing, and the like. Zero padding that fills an area outside the tomographic image with a pixel value of zero may be executed.

In the embodiment, a starting point of boundary search is a deepest point on each of the search routes, and boundary search is sequentially advanced from the starting point to a shallower point. In a breast tomographic image, a boundary image clearly appears between a mammary gland image and a pectoralis major image. A back side (deep side) of the boundary image is a low-luminance region having substantially uniformity. Based on these properties or characteristics, the boundary search is sequentially executed from a deep place to a shallow place. In the embodiment, an observation target is a mammary gland image, and the mammary gland image is present at a front side; that is, a shallow side of the boundary image.

The exclusion processor 38 executes processing of excluding a detection point satisfying an exclusion condition as an invalid detection point among a plurality of detection points configuring a detection point row. Thereby, a plurality of effective detection points are left. The detection point row is reconstructed by the plurality of effective detection points.

The approximate straight line generator 40 generates an approximate straight line based on a plurality of effective detection points. In this case, for example, a least squares method or the like is used. A region at an upper side of the approximate straight line may be defined as a region of interest (ROI). Image analysis is executed in the region of interest. A lower side of the region of interest may be defined by a curve approximating a plurality of effective detection points. Spatial smoothing may be applied to a boundary point row formed of a plurality of boundary points. Further, temporal smoothing may be applied to the boundary point row. Spatial smoothing and temporal smoothing may be applied to the lower side of the region of interest.

The angle calculator 42 calculates an intersection angle of the approximate straight line and a horizontal line as an inclination angle θ. An intersection angle between a vertical line and the approximate straight line may be calculated. The inclination angle may be calculated directly from a plurality of effective detection points simulating a boundary image without generating the approximate straight line.

The average depth calculator 43 calculates an average depth d concerning the approximate straight line. For example, the average depth d may be calculated by averaging y-coordinates of a plurality of effective detection points, or the average depth d may be calculated by averaging y-coordinates of a plurality of pixels configuring the approximate straight line. The average depth d may be calculated as a middle point between the y-coordinates of both ends of the approximate straight line. The average depth d is referred to in variable setting of a threshold θ1.

The threshold setting device 44 sets the threshold θ1 which is compared with the inclination angle θ. The threshold θ1 is set based on designation of the user, or is set automatically and adaptively. In the illustrated example configuration, the threshold θ1 can be variably set based on the depth of the approximate straight line.

The generation controller 46 controls an operation of the support image generator 48, and more specifically controls the support image generator 48 so that a support image having an alert mode is generated when the inclination angle θ exceeds the threshold θ1, whereas a support image having a non-alert mode is generated when the inclination angle θ is less than or equal to the threshold θ1. A support image is generated in either or both of a case where a probe contact posture is inappropriate and a case where the probe contact posture is appropriate.

The support image generator 48 generates a support image that supports a probe operation. A display form of the support image is changed in response to the inclination angle as described above. The above-described alert mode is a mode that calls attention to the user, and for example, a certain figure is displayed in a conspicuous color, displayed with high luminance, displayed in a prominent form, or displayed in an enlarged manner.

Information on the inclination angle is also given to the determining unit shown in FIG. 1. The determining unit makes the CAD function invalid when the inclination angle θ exceeds the threshold θ1, and makes the CAD function effective when the inclination angle θ is less than or equal to the threshold θ1. The threshold for controlling generation of the support image may differ from the threshold for controlling the CAD function to be turned on/off.

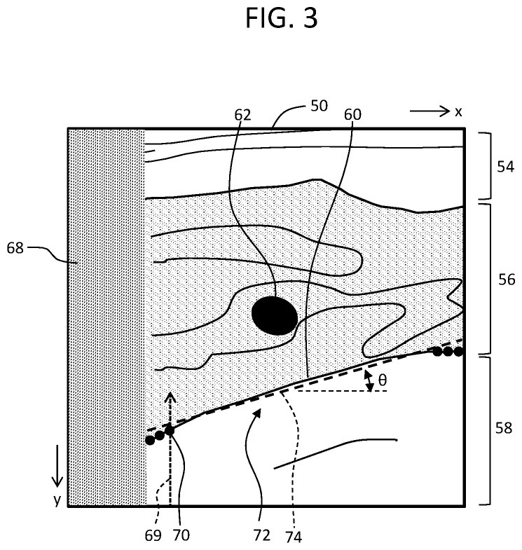

FIG. 3 shows a tomographic image 50 generated by ultrasonic diagnosis of a breast. The tomographic image 50 is a B-mode tomographic image displayed in real time. “x” indicates a horizontal direction (lateral direction), which is an electron scanning direction in the embodiment. “y” indicates a vertical direction (longitudinal direction), which is a depth direction in the embodiment.

The tomographic image 50 includes a fat layer image 54, a mammary gland image (mammary gland layer image) 56, and a pectoralis major image 58. Further, the tomographic image 50 includes a linear boundary image 60 between the mammary gland image 56 and the pectoralis major image 58. In the illustrated example, a tumor (tumor image) 62 is included in the mammary gland image 56, and a shadow 68 is included in the tomographic image 50. The shadow 68 occurs when the probe is not appropriately applied to the breast, such that partial reduction in the degree of close contact occurs between the wave transmission and reception surface and the breast surface, or when a part where the mammary gland is not sufficiently spread occurs because the pressing force by the probe becomes insufficient, such that ultrasound does not sufficiently reach the back side of the part. When necessary pressing or necessary stretching of the mammary gland cannot be performed, an unclear part other than the shadow is easily generated. In the illustrated example, the boundary image 60 inclines significantly; more specifically, a right side thereof rises, whereas a left side thereof lowers.

A tomographic image including many artifacts such as a shadow is not suitable for interpretation, and when CAD is applied to the tomographic image, erroneous recognition of an abnormal site easily occurs. For example, when CAD is applied to the tomographic image 50 shown in FIG. 3, a specific portion in the shadow may be erroneously recognized as an abnormal site.

In order to reduce artifacts, and enhance quality of the tomographic image, a relatively soft mammary gland is desirably sandwiched between the wave transmission and reception surface of the probe and the pectoralis major; in other words, the state where the mammary gland is stretched in the horizontal direction is desirably caused. In other words, it is necessary to adjust the contact posture of the probe so that the wave transmission and reception surface of the probe and the boundary image become closer to a parallel relation while pressing the probe against the breast. The above-described support image is an image for supporting the probe operation in this manner, and more specifically, is information indicating the appropriateness/inappropriateness of the inclination angle (probe contact posture) of the boundary image 60.

The inclination angle of the boundary image 60 is calculated as follows. First, a plurality of search routes 69 are equidistantly set parallel with the y-direction in the tomographic image. On each of the search routes 69 a search is conducted for an edge corresponding to the boundary image 60, and a detection point of the edge is made a boundary point 70. A boundary point row 72 is configured by a plurality of boundary points in an x-direction. An approximate straight line 74 is generated based on the boundary point row 72, and the inclination angle θ of the boundary image 60 is calculated as the intersection angle which is formed by the approximate straight line 74 with a horizontal line. In reality, after exclusion processing is applied to the boundary point row 72, the approximate straight line is calculated based on a boundary point row after the exclusion processing.

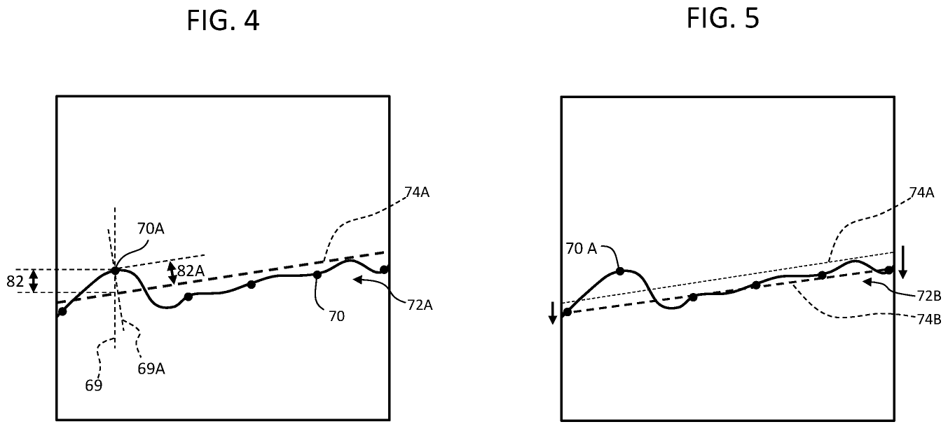

FIG. 4 specifically shows a generation method of the approximate straight line. In the illustrated example, a plurality of boundary points 70 are detected on the boundary image, and a boundary point row 72A is configured by the plurality of boundary points 70. In the embodiment, among the plurality of boundary points 70, a boundary point satisfying a predetermined exclusion condition is excluded as an invalid boundary point. For example, an approximate straight line (temporary approximate straight line) 74A is generated by a least squares method based on the boundary point row 72A, and a boundary point satisfying an exclusion condition based on the approximate straight line 74A is determined as the invalid boundary point.

For example, at each boundary point, a y-direction distance (vertical distance) between the boundary point and the approximate straight line 74A is calculated, and a boundary point that causes a maximum distance is determined as the invalid boundary point. In that case, n (n is an integer greater than or equal to 1) boundary points may be respectively determined as invalid boundary points in the order of distance. Alternatively, all boundary points with distances equal to or greater than a predetermined value may be determined as invalid boundary points. In the example shown in FIG. 4, for example, on the search route 69, a y-direction distance 82 between a boundary point 70A and the approximate straight line 74A is a maximum distance, and the boundary point 70A that causes the maximum distance is determined as the invalid boundary point. A distance 82A on a line 69A orthogonal to the approximate straight line 74A may be calculated.

FIG. 5 shows a boundary point row 72B after the exclusion processing is applied to the boundary point 70A. An approximate straight line 74B is recalculated based on the boundary point row 72B. The approximate straight line 74B is a straight line different from the approximate straight line 74A obtained first, and is not affected by the boundary point 70A. By the exclusion processing, it becomes possible to generate a more accurate approximate straight line by excluding an influence of a local change. Generation of the approximate straight line and the exclusion processing may be executed repeatedly.

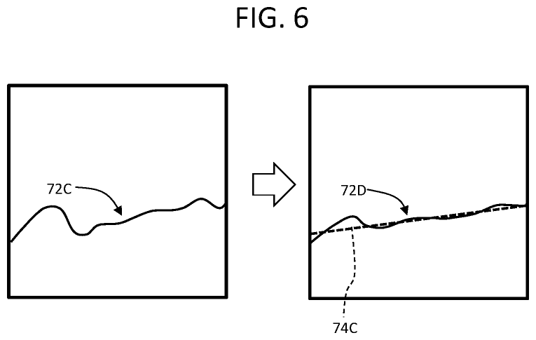

As shown in FIG. 6, spatial smoothing is applied to a boundary point row 72C, and an approximate straight line 74C may be generated based on a boundary point row 72D after smoothing. In FIG. 6, in order to express a difference before and after smoothing clearly, the boundary point rows before and after smoothing are each represented by one line.

Before or after spatial smoothing, temporal smoothing may be applied to the boundary point row. By temporary smoothing, it becomes possible to restrain the form of the approximate straight line from drastically changing in frame units, and it becomes possible to stabilize the inclination angle which is calculated. As a matter of course, the function of spatial smoothing may be turned off while the ultrasound probe is moved.

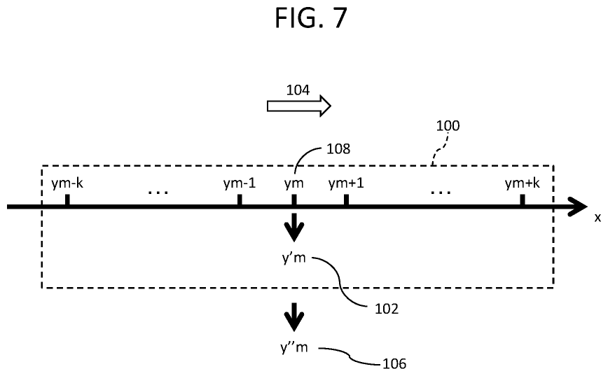

FIG. 7 illustrates a smoothing method. An x-direction is a horizontal direction, and on an x-axis, y-coordinates of a plurality of boundary points detected on a plurality of search routes are shown. Among them, y-coordinates (ym−k to ym to ym+k) included in a fixed section 100 with an x-coordinate of interest (y-coordinate is ym) (see reference numeral 108) as a center are specified, a spatial average value y′m of these y-coordinates is calculated (see reference numeral 102), and the spatial average value y′m is assigned to the x-coordinate of interest.

The above-described processing is repeatedly executed while the section 100 is moved (see reference numeral 104). A weighted average or the like may be used in place of the simple average. Further, in individual x-coordinates, y-coordinates are smoothed in a time axis direction, a temporal and spatial average value y″m (see reference sign 106) is calculated, and the temporal and spatial average value y″m may be assigned to the individual x-coordinates.

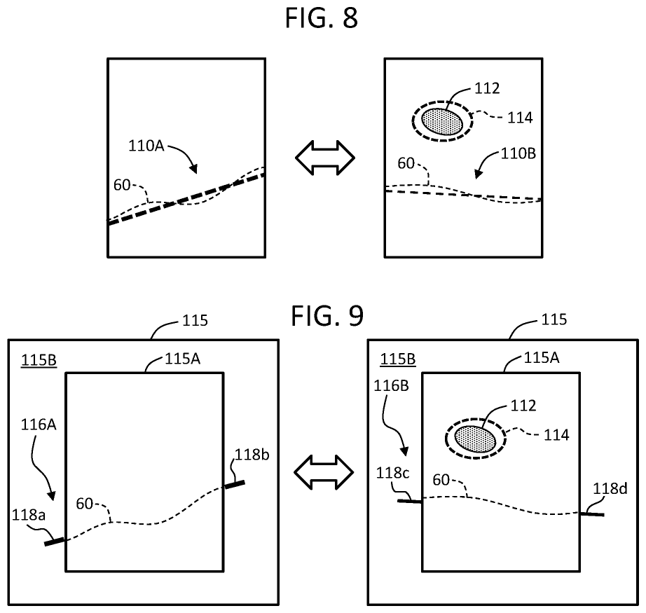

FIG. 8 shows a first example of the support image. A support image 110A having an alert mode is shown on a left side of FIG. 8, and a support image 110B having a non-alert mode is shown on a right side of FIG. 8. The support image 110A is configured by a red line indicating an approximate straight line. The support image 110B is configured by a green line indicating an approximate straight line. Both lines are displayed by being superimposed on boundary images 60. The respective lines are displayed as semitransparent lines in order to enable observation of the boundary images 60. When the support image 110A is displayed in the process of a probe operation, the user can recognize that the probe contact posture is inappropriate through the observation. Thereafter, at a time point when the support image 110B is displayed, the user can recognize that the probe contact posture is made appropriate. The boundary point row may be displayed instead of the line.

In the embodiment, the CAD function is automatically turned off when the inclination angle exceeds the threshold, and the CAD function is automatically turned on when the inclination angle is equal to or smaller than the threshold. In the example shown in FIG. 8, a tumor 112 included in the tomographic image on the right side is surrounded by a marker 114; that is, an abnormal site (more accurately, an abnormal site candidate) is automatically marked. The support image may be generated by painting a lower side from the approximate straight line translucently.

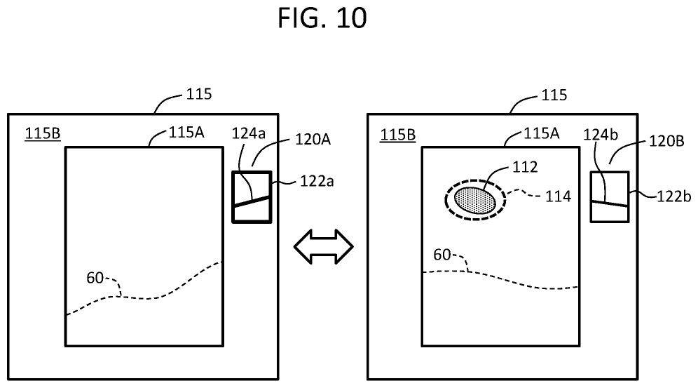

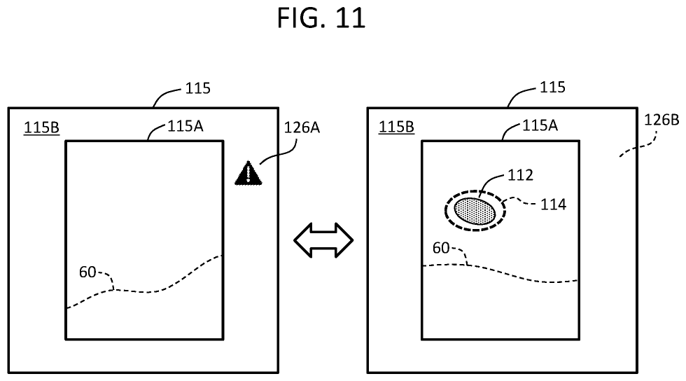

FIG. 9 shows a second example of the support image. Note that the elements already explained are assigned the same reference numerals, and repeated explanation thereof is omitted. This similarly applies to elements shown in FIG. 10 and FIG. 11 that are described later.

In FIG. 9, a support image 116A having an alert mode is shown on a left side, and a support image 116B having a non-alert mode is shown on a right side. Display images 115 each have a tomographic image display area 115A and a surrounding area 115B around the tomographic image display area 115A, and the support images 116A and 116B are displayed in the surrounding areas 115B.

More specifically, the support image 116A is configured by two markers 118a and 118b that are displayed on a line that is conceived by extrapolating an approximate straight line, and these markers have a red color, for example. The two markers 118a and 118b are displayed in the surrounding area 115B. The support image 116B is configured by two markers 118c and 118d that are displayed on a line conceived by extrapolating an approximate straight line as described above, and these markers have a green color, for example. The two markers 118c and 118d are displayed in the surrounding area 115B. The user is informed of an alert state by display of the red color, in contrast with the green color.

According to the second example, the support images 116A and 116B do not overlap the boundary image 60, and therefore there is provided an advantage that observation of the boundary image 60 is not hindered by the support images 116A and 116B.

FIG. 10 shows a third example of the support image. A support image 120A having an alert mode is shown on a left side of FIG. 10, and a support image 120B having a non-alert mode is shown on a right side of FIG. 10. Display images 115 each have a tomographic image display area 115A, and a surrounding area 115B around the tomographic image display area 115A, and the support images 120A and 120B are displayed in the surrounding areas 115B.

The individual support images 120A and 120B are configured by frames 122a and 122b simulating modes of the tomographic images, and lines 124a and 124b simulating approximate straight lines 124a and 124b. The support image 120A has a red color, for example, and the support image 120B has a green color, for example. It may be the case that only the support image 120B is displayed. According to the third example, a depth position and a gradient of the boundary image can be easily recognized visually.

FIG. 11 shows a fourth example of the support image. A support image 126A that is displayed when the inclination angle exceeds the threshold is shown on a left side of FIG. 11. The display image 115 has the tomographic image display area 115A and the surrounding area 115B around the tomographic image display area 115A, and the support image 126A is displayed in the surrounding area 115B. The support image 126 is configured by an alert symbol formed of a red triangle. The display image 115 which is displayed when the inclination angle is less than or equal to the threshold is shown on a right side of FIG. 11. No support image is displayed in the surrounding area 115B shown by reference numeral 126B.

In the second example to the fourth example, CAD functions are automatically turned off when the inclination angles exceed the threshold, and the CAD functions are automatically turned on when the inclination angles are less than or equal to the threshold, as in the first example. Tumors 112 included in the tomographic images on the right side are marked by markers 114.

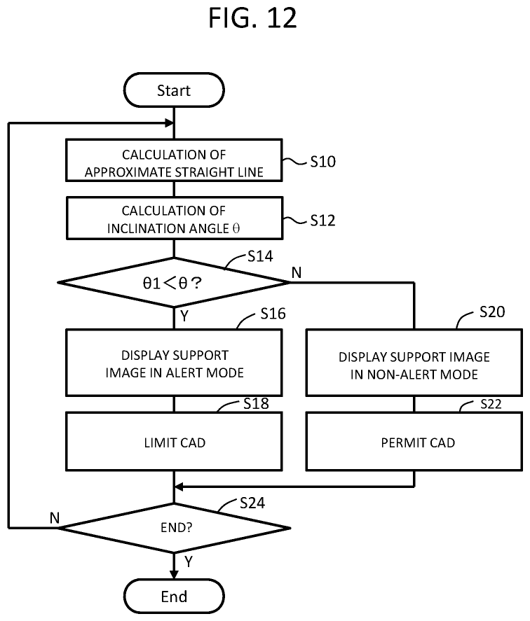

FIG. 12 shows an operation example (in particular, an operation example concerning display) of the ultrasound diagnostic apparatus shown in FIG. 1 as a flowchart. In S10, a boundary point row is generated based on a boundary image in a tomographic image, and an approximate straight line is generated based on the boundary point row. The above-described exclusion processing may be applied in the process. In S12, the inclination angle θ of the approximate straight line is calculated. In the embodiment, the inclination angle θ is an intersection angle of the approximate straight line and a horizontal line. In S14, the inclination angle θ is compared with the threshold θ1. When the inclination angle θ exceeds the threshold θ1, a support image having an alert mode is displayed in S16, and subsequently, in S18, CAD is limited. For example, display of a CAD result is prohibited. On the other hand, when the inclination angle θ is determined as less than or equal to the threshold θ1 in S14, a support image having a non-alert mode is displayed in S20, and subsequently, CAD is permitted in S22.

As above, according to the operation of the embodiment, the support image having the alert mode is displayed when the probe contact posture is inappropriate, so that the user can recognize the state through observation of the support image, and can perform an operation of changing the posture of the probe based on this information. When the support image having a non-alert mode is displayed in that process, the user can confirm that the probe contact posture is appropriate through observation of the support image. Further, according to the embodiment, the function of the CAD can be exhibited only when the probe contact posture is appropriate, and therefore, occurrence of erroneous recognition of an abnormal site can be prevented or reduced. It may be the case that the support image is displayed only when the inclination angle θ exceeds the threshold θ1.

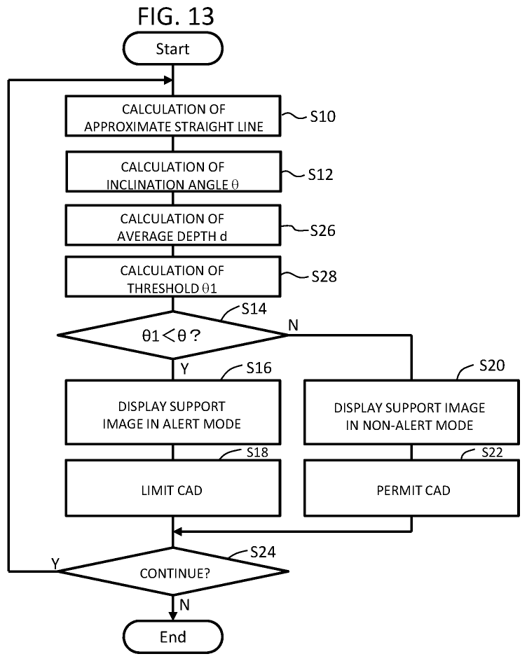

FIG. 13 shows another operation example as a flowchart. Note that the same steps as the steps shown in FIG. 12 are assigned with the same reference signs and repeated explanation thereof is omitted.

In the operation example shown in FIGS. 13, S26 and S28 are added between S12 and S14. In S26, based on an approximate straight line, the average depth d thereof is calculated. In S28, the threshold θ1 is adaptively set based on the average depth d. More specifically, as the average depth d decreases, the threshold θ1 is more relaxed; that is, the threshold θ1 is increased. Conversely, as the average depth d increases, the threshold θ1 is made more stringent; that is, the threshold θ1 is decreased. For example, the threshold θ1 is calculated from θ1=θ0−k·d, by using a coefficient k and a reference value θ0. In S14, the inclination angle θ is compared with the threshold value θ1 which is adaptively set.

When the probe is applied to an end portion of a breast, the boundary image tends to appear in a shallow part on the tomographic image, and the boundary tends to incline. Thickness of a mammary gland varies from patient to patient, and it is found that the boundary tends to be inclined when the mammary gland is thin. Therefore, when the boundary is present in a shallow part, the threshold is increased to relax the threshold, whereas when the boundary is present in a deep part, the threshold is decreased to make the threshold stringent. According to the operation example shown in FIG. 13, it becomes possible to avoid a problem that the threshold θ1 is too stringent when the probe is brought into contact with the end portion of the breast and ultrasound examination is performed, for example.

In the above-described embodiment, turning the CAD function on/off is controlled based on the inclination angle, but turning an elastography image on/off may be controlled based on the inclination angle in elastography. Alternatively, the elastography image may be analyzed when the inclination angle is less than or equal to the threshold.

In the above-described embodiment, it may be the case that calculation of the approximate straight line is not performed when, in detection of the boundary image, the number of detected boundary points is extremely small, or when an error amount between the approximate straight line and a plurality of boundary points is extremely large. In the configuration in which the support image is not generated, control of turning the image analysis on/off may be performed based on the inclination angle of the boundary image. Modified examples can also be considered, in which the technique according to the above-described embodiment is applied to tissues other than the breast.

1. An ultrasound diagnostic apparatus, comprising:
a probe that is brought into contact with a breast, and outputs a reception signal by transmission and reception of ultrasonic waves to and from the breast;
an image generating unit that, based on the reception signal, generates an ultrasound image including a mammary gland image, a pectoralis major image, and a boundary image between the mammary gland image and the pectoralis major image;
an inclination angle calculating unit that, based on the ultrasound image, calculates an inclination angle of the boundary image; and
a support image generating unit that generates a support image that supports an operation of the probe, based on the inclination angle of the boundary image.

2. The ultrasound diagnostic apparatus according to claim 1, further comprising:
a determining unit that, based on the inclination angle of the boundary image, determines inappropriateness concerning a contact posture of the probe,
wherein a user is informed of the inappropriateness through the support image.

3. The ultrasound diagnostic apparatus according to claim 2,
wherein the determining unit determines the inappropriateness when the inclination angle exceeds a threshold, and
a threshold setting unit that changes the threshold in accordance with a depth of the boundary image is provided.

4. The ultrasound diagnostic apparatus according to claim 3,
wherein the threshold setting unit increases the threshold with decrease of the depth of the boundary image.

5. The ultrasound diagnostic apparatus according to claim 1,
wherein the inclination angle calculating unit comprises
a generator that generates an approximate straight line based on the boundary image, and
a calculator that calculates an intersection angle of the approximate straight line to a horizontal direction, as the inclination angle.

6. The ultrasound diagnostic apparatus according to claim 5,
wherein the generator
sets a plurality of search routes to cross the boundary image,
performs boundary search from a deep side to a shallow side on the respective search routes to specify boundary points, and
generates the approximate straight line based on a plurality of boundary points specified on the plurality of search routes.

7. The ultrasound diagnostic apparatus according to claim 6,
wherein the generator
excludes an invalid boundary point that satisfies an exclusion condition among the plurality of boundary points to specify a plurality of effective boundary points, and
generates the approximate straight line based on the plurality of effective boundary points.

8. The ultrasound diagnostic apparatus according to claim 1, comprising:
an analyzing unit that searches for an abnormal site in the ultrasound image; and
a control unit that limits an operation of the analyzing unit based on the inclination angle of the boundary image.

9. A display method, comprising the steps of:
calculating an inclination angle of a boundary image, based on an ultrasound image including a mammary gland image, a pectoralis major image, and the boundary image between the mammary gland image and the pectoralis major image;
generating a support image that supports an operation of a probe contacting a breast, based on the inclination angle of the boundary image; and
displaying the support image.

10. A non-transitory storage medium storing a program,
Wherein the program comprising:
a function of calculating an inclination angle of a boundary image, based on an ultrasound image including a mammary gland image, a pectoralis major image, and the boundary image between the mammary gland image and the pectoralis major image, and
a function of generating a support image that supports an operation of a probe contacting a breast, based on the inclination angle of the boundary image.
